(19) United States
(12) Patent Application Publication
(10) Pub. No.: US 2026/0061259 A1
(43) Pub. Date: Mar. 5, 2026

(54) INSTRUCTIONAL ASSESSMENT INTERFACE

(71) Applicant: NeckCare hf., Kopavogur (IS)

(72) Inventors: Darri Steinn Konn KONRADSSON, Alftanes (IS); Jona Marin OLAFSDOTTIR, Hafnarfjordur (IS); Tomas PALSSON, Reykjavík (IS); Alexander Josep BLONDAL, Kopavogur (IS); Birkir Orn KARLSSON, Kopavogur (IS)

(21) Appl. No.: 19/312,913

(22) Filed: Aug. 28, 2025

Related U.S. Application Data
(60) Provisional application No. 63/689,237, filed on Aug. 30, 2024.
(60) Provisional application No. 63/805,728, filed on May 14, 2025.

(51) Int. Cl. A63B 24/00; A63B 71/06; G06F 3/01; G06T 7/00; G06T 19/20; G16H 15/00

(52) U.S. Cl. CPC A63B 24/0062; A63B 71/0622; G06F 3/011; G06T 7/0012; G06T 19/20; G16H 15/00; A63B 2024/0068; G06T 2200/24; G06T 2207/30041; G06T 2207/30196; G06T 2210/41; G06T 2219/2012

(57) ABSTRACT

A system for evaluating a condition affecting musculoskeletal or neurological health of a user using an assessment application with an improved instructional interface and exercise module. The instructional interface automatically displays an instructional image corresponding to an activity of an assessment. After the assessment application detects the activity being performed by the user, the instructional interface foregrounds the instructional image corresponding to the activity. After automatically determining an endpoint for the activity, the assessment application subsequently detects another activity being performed by the user. Upon detecting the other activity, the instructional interface foregrounds another instructional image corresponding to the other activity. The assessment application then automatically determines another endpoint for the other activity and generates a report based on performance of the user for the assessment and/or exercise.

### FIELD OF THE DISCLOSURE

The disclosure relates to an instructional interface and exercise module of an assessment application for performing computer-prompted activities, in particular, related to cervical-spinal health.

### BACKGROUND

Existing sensorimotor control tests, such as the Butterfly test for assessing musculoskeletal movement, have been shown to provide sufficient diagnostic accuracy for differentiating between patients with cervical spine impairment and healthy individuals. An exemplary method for assessment and graded training of sensorimotor functions is provided in international application No. PCT/IS2010/000010, filed on Jul. 7, 2010, and published as WO 2011/004403 A1 on Jan. 13, 2011, which is incorporated herein by reference.

However, existing systems for assessing sensorimotor functions and other physical functions include risks of operator error. For example, the steps required by operators to conduct assessments can include setting up manual start and end points, which requires the operator or trained provider to identify when an activity begins and ends. Additionally, while existing systems (or trained providers) may initially provide instructions for how to complete an activity, such instructions are not visually present during the activity and are unadaptable to change because of the scripted assessment. As such there is a need for a more flexible, intuitive instructional assessment interface that avoids operator error.

### SUMMARY

The inventors of the present disclosure developed an improved instructional interface for completing computer-guided assessments to evaluate various conditions of an individual, including but not limited to musculoskeletal, neuromuscular, or neurological health conditions of the individual. The present disclosure provides a solution for assessing sensorimotor functions and other physical functions that also mitigates the risks of operator error. The disclosed instructional interface includes adaptable instructions that are visibly presented as instructional images to the user during performance of an activity, wherein the instructional images are automatically updated to reflect the current activity that the user is performing. In other words, the system and method are adaptable to the actions of the user and offer greater flexibility in completing activities to evaluate various musculoskeletal and neuromuscular conditions.

In an embodiment, the disclosure relates to a system for evaluating a condition affecting musculoskeletal or neurological health of a user using an assessment application. The system comprises a first computing unit having the assessment application, a processor, one or more hardware storage devices, and a display. The assessment application includes an instructional interface having at least one assessment with at least one activity for the user to perform. The system further includes a tracking device that is communicatively connected to the first computing unit and configured to observe physical assessment performance of the user during the at least one assessment.

The one or more hardware storage devices store instructions that are executable by the system to perform various actions, including initiating the assessment application and obtaining kinematic data corresponding to the user in a three-dimensional space. The assessment application automatically determines a starting point for the at least one assessment based on the kinematic data. The instructional interface displays at least one instructional image corresponding to at least one activity of the at least one assessment. After the assessment application detects enactment of an activity (e.g., first activity) being performed by the user, the instructional interface foregrounds a first instructional image corresponding to the first activity. The assessment application then automatically determines a first endpoint for the first activity and subsequently detects a second activity being performed by the user. Upon detecting the second activity, the instructional interface foregrounds a second instructional image corresponding to the second activity. Following this, the assessment application automatically determines a second endpoint for the second activity and generates a report based on performance of the user for the at least one assessment.

The first instructional image can be different from the second instructional image. In cases where the user sequentially completes the same activity, the first instructional image is the same as the second instructional image. Or, in other words, the first instructional image remains present during completion of multiple activities that are the same. In an embodiment, the first instructional image and second instructional image are simultaneously displayed on the display, such as a monitor or screen. Optionally, in embodiments where the first and second instructional images are displayed together, the first instructional image can be muted when the second instructional image is foregrounded to provide greater visual emphasis or cue to the individual. In an alternative embodiment, the first instructional image is switched or replaced by the second instructional image after the second activity is detected as being performed by the user.

In an embodiment, the present disclosure relates to a method for evaluating a condition affecting musculoskeletal or neurological health of a user using a computing unit having an assessment application, a processor, one or more hardware storage devices, and a display, wherein the assessment application includes an instructional interface and is arranged to record physical assessment performance of the user during at least one assessment completed with the instructional interface.

The method comprises the following steps: initiating the assessment application, obtaining kinematic data corresponding to the user in a three-dimensional space, automatically determining a starting point for the at least one assessment based on the kinematic data, displaying at least one instructional image corresponding to at least one activity of the at least one assessment, detecting a first activity being performed by the user, foregrounding a first instructional image corresponding to the first activity, automatically determining a first endpoint for the first activity, detecting a second activity being performed by the user, foregrounding a second instructional image corresponding to the second activity, automatically determining a second endpoint for the second activity, and generating a report based on performance of the user for the at least one assessment.

In another aspect related to motion control, the present disclosure relates to a method for evaluating a condition affecting musculoskeletal or neurological health of a user using a computing unit having an assessment application, a processor, one or more hardware storage devices, and a display, wherein the assessment application includes an instructional interface and is arranged to record physical assessment performance of the user during at least one assessment completed with the instructional interface.

The method comprises the following steps: initiating the assessment application, obtaining kinematic data corresponding to the user in a three-dimensional space, automatically determining a starting point for a movement control assessment based on the kinematic data, displaying at least one instructional image or text corresponding to at least one activity of the movement control assessment, detecting a first activity being performed by the user, the first activity including a moving indicator that follows a computer-generated path and a cursor controllable by the user for performing the first activity, visually indicating movement accuracy performance of the user during execution of the first activity by a change in color of the moving indicator and based on deviation of the cursor from the moving indicator, automatically determining an endpoint corresponding to the first activity, and generating a report based on performance of the user for the at least one assessment.

In another aspect of the present disclosure, the assessment application relates to an exercise module for training and rehabilitation for the user to treat the condition. One skilled in the art will recognize that features of the instructional interface in the present disclosure likewise apply to the exercise module.

These and other features, aspects, and advantages of the present disclosure will help better understand the following description, appended claims, and accompanying drawings.

### BRIEF DESCRIPTION OF THE DRAWINGS

In order to describe the manner in which the advantages and features of the systems and methods described herein can be obtained, a more particular description of the embodiments briefly described above will be rendered by reference to specific embodiments thereof which are illustrated in the appended drawings. Understanding that these drawings depict only typical embodiments of the systems and methods described herein, and are not therefore to be considered to be limiting of their scope, certain systems and methods will be described and explained with additional specificity and detail through the use of the accompanying drawings.

FIG. 1 illustrates a system for executing a computer-directed assessment for evaluating a condition affecting musculoskeletal or neuromuscular health.

FIG. 2 illustrates a system of devices and a service for performing the computer-directed assessment.

FIGS. 3A-3B illustrate an instructional interface for carrying out at least one computer-prompted activity related to range of motion.

FIG. 4 illustrates an instructional interface for having a foregrounded instructional image.

FIG. 5 illustrates an instructional interface for carrying out a computer-prompted activity related to joint position error.

FIG. 6 illustrates a graphical scorecard corresponding to performance of at least one computer-prompted activity.

FIG. 7 illustrates a graphical representation of plane association relating to kinematic data of a user in three-dimensional space.

FIG. 8 illustrates an instructional interface for carrying out a computer-prompted activity related to motion control.

FIG. 9 illustrates a flowchart of a method for implementing the computer-directed assessment program using an instructional interface with a set of instructional images.

FIG. 10 illustrates a flowchart of a method for implementing the computer-directed assessment program including automatically updating instructional images to align with corresponding computer-prompted activities.

FIG. 11 illustrates a flowchart of a method for implementing the computer-directed assessment program including simultaneously completing a joint position error assessment and a range of motion assessment.

FIG. 12 illustrates a flowchart of a method for implementing the computer-directed assessment program including indicating directional accuracy of a user during execution of at least one computer-prompted activity.

FIG. 13 illustrates an exemplary interface for an exercise module of the assessment application implemented by the system.

FIG. 14A illustrates an exemplary user interface for the Butterfly exercise.

FIG. 14B illustrates an exemplary user interface displaying results of the Butterfly exercise.

FIG. 14C illustrates an exemplary playback user interface for displaying the performance of the Butterfly exercise.

FIG. 15A illustrates an exemplary user interface for the joint position error exercise.

FIG. 15B illustrates an exemplary user interface displaying results of the joint position error exercise.

FIG. 15C illustrates an exemplary playback user interface for displaying the performance of the joint position error exercise.

FIG. 16A illustrates an exemplary user interface for the controlled articular rotations exercise.

FIG. 16B illustrates an exemplary user interface displaying results of the controlled articular rotations exercise.

FIG. 17 illustrates an exemplary user interface for the set point in range exercise.

The drawing figures are not necessarily drawn to scale. Instead, they are drawn to provide a better understanding of the components and are not intended to be limiting in scope but providing exemplary illustrations.

### Definitions

For ease of understanding the disclosed embodiments of the present disclosure and associated method and system elements, a description of a few terms is necessary.

The term ‘assessment program’ or ‘assessment application’ generally refers to an interactive software having at least one assessment (e.g., Butterfly test, range of motion assessment, relocation assessment, rehabilitation exercise) and at least one activity (e.g., flexion, extension, rotation) conducted using a computer and performed by an individual, intended to evaluate a certain physical function, wherein the performance of the at least one assessment, based on the at least one activity, may be used to generate a report for evaluating a condition affecting musculoskeletal or neurological health of the individual and also to classify the individual as having an impairment or condition. The assessment program may be related to motion, strength, balance, etc. and may also be used as a rehabilitative exercise for treating a condition. The disclosed activities and assessments may be configured as trials, assessments, rehabilitative activities, and/or treatment tasks.

The term ‘computer’ or ‘computing unit’ may include any device that comprises at least one processor and that electronically executes one or more programs, such as a user interface program and/or software program, and may include personal computers, laptop computers, servers, portable media players, hand-held devices, cellular phones, microprocessor-based programmable consumer electronic and/or appliances, and other similar electronic devices that include circuitry for wirelessly sending and/or receiving information.

The term ‘computer-directed assessment,’ ‘instructional interface,’ ‘exercise module,’ or ‘assessment’ may generally refer to one or more predefined tests and activities described in the present disclosure. Non-limiting examples of the computer-directed assessment include the Butterfly test (including variations of a visible path, small/large path, and non-centered path), range of motion assessment, joint position error test with and without offset, controlled articular rotations (CARs), set points in range (start in center and then reach targets at different locations with the cursor), zigzag test and similar (trace a visible path with the cursor at a self-selected speed), smooth pursuit neck torsion test, gaze stability (e.g., maintaining gaze while moving the head), saccadic eye movements, and discrete eye-head coordination. The assessment may also refer to rehabilitation exercise and training tasks for rehabilitative and performance enhancement or instructional purposes, respectively.

The term ‘impairment’ refers to a condition, disability, injury, or symptomatic state affecting the muscular, skeletal, and/or nervous systems of an individual.

The term ‘user’ refers to a user of the system or, more specifically, a patient or person using the computer-directed assessment program to complete at least one assessment and activity. In the context of the instructional interface being displayed to the user, such may also be displayed to a provider. Thus, the term ‘user’ may also refer to a provider using the system for assessing a patient's performance.

The term ‘movement control’ refers to sensorimotor control, neuromuscular control, and the like.

Unless otherwise specified, the term ‘network’ refers to one or more data links, e.g., comprising a database, that enable the wired or wireless transport of electronic data between computer systems and/or modules and/or other electronic devices.

The term ‘processor’ or ‘processing unit’ refers to one or more devices, circuits, and/or processing cores configured to process data, such as computer program instructions, and includes personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. Unless otherwise stated, references to a first processor may also apply to a second processor and vice versa.

The term ‘provider’ may include a medical or healthcare professional (e.g., such as a doctor, a nurse, a therapist, and the like), an exercise professional (e.g., such as a coach, a trainer, a nutritionist, and the like). As used herein, and without limiting the foregoing, a ‘healthcare professional’ may be a human being, a robot, a virtual assistant, or an artificially intelligent entity, such entity including a software program, integrated software and hardware, or hardware alone.

The term ‘service’ refers to an automated program that is tasked with performing different actions based on input. As used herein, the terms ‘executable module,’ ‘executable component,’ ‘component,’ ‘module,’ ‘service,’ or ‘engine’ can refer to hardware processing units or to software objects, routines, or methods that may be executed on or with the system.

The term ‘software’ generally refers to computer-executable instructions, code, data, applications, programs, program modules, or the like maintained in or on any form or type of computer-readable media that is configured for storing computer-executable instructions or the like in a manner that is accessible to a computing unit.

As used herein, reference to any type of machine learning or artificial intelligence may include any type of machine learning algorithm or device, convolutional neural network(s), multilayer neural network(s), recursive neural network(s), deep neural network(s), decision tree model(s) (e.g., decision trees, random forests, and gradient boosted trees) linear regression model(s), logistic regression model(s), support vector machine(s) (SVM), artificial intelligence device(s), or any other type of intelligent computing system. Any amount of training data may be used (and perhaps later refined) to train the machine learning algorithm to dynamically perform the disclosed operations.

### DETAILED DESCRIPTION OF VARIOUS EMBODIMENTS

A better understanding of different embodiments of the disclosure may be had from the following description read with the accompanying drawings in which reference characters refer to like elements. While the disclosure is susceptible to various modifications and alternative constructions, certain illustrative embodiments are in the drawings and are described below. It should be understood, however, that there is no intention to limit the disclosure to the embodiments disclosed; on the contrary, the intention covers all modifications, alternative constructions, combinations, and equivalents falling within the spirit and scope of the disclosure.

With respect to the use of plural and/or singular terms herein, those skilled in the art may translate the terms from the plural to the singular and/or from the singular to the plural as is appropriate to the context and/or application. The various singular/plural permutations may be expressly set forth herein for sake of clarity.

It will be understood that unless a term is defined to possess a described meaning, there is no intent to limit the meaning of such term, either expressly or indirectly, beyond its plain or ordinary meaning.

The disclosed system is based on the functional and statistical interpretation of objective innovative data derived from measurements of musculoskeletal movements of individuals recorded in conjunction with one or more computer-directed assessments. The disclosed solution provides a system and method for evaluating a condition affecting musculoskeletal or neurological health of a user using an assessment application that avoids operator error and automatically determines starting points, neutral (resting) points, and endpoints for each activity and assessment.

### Exemplary Embodiment to Objectively Assess and Diagnose Cervical Impairment

Affecting nearly two-thirds of the general population at least once in their life, neck pain is a growing healthcare concern. Common causes for this condition can include whiplash, a blow to the head, strenuous working conditions, or sustained poor posture. For example, professionals who spend many hours hunched over their workspace-such as surgeons and dentists-frequently develop neck pain. Those who wear heavy protective helmets, including athletes, jet and helicopter pilots, warriors and firefighters, are also at risk.

The techniques clinicians currently use to assess neck impairment have significant drawbacks because a majority of them rely on cumbersome tests, such as manually operated range-of-motion (ROM) assessments, which make it difficult to gauge the extent of an injury or track progress during therapy. These manually operated ROM assessments are also prone to operator error and may lack consistency in implementation and practice. Some techniques also require labor-intensive manual procedures involving a laser pointer attached to the patient's head.

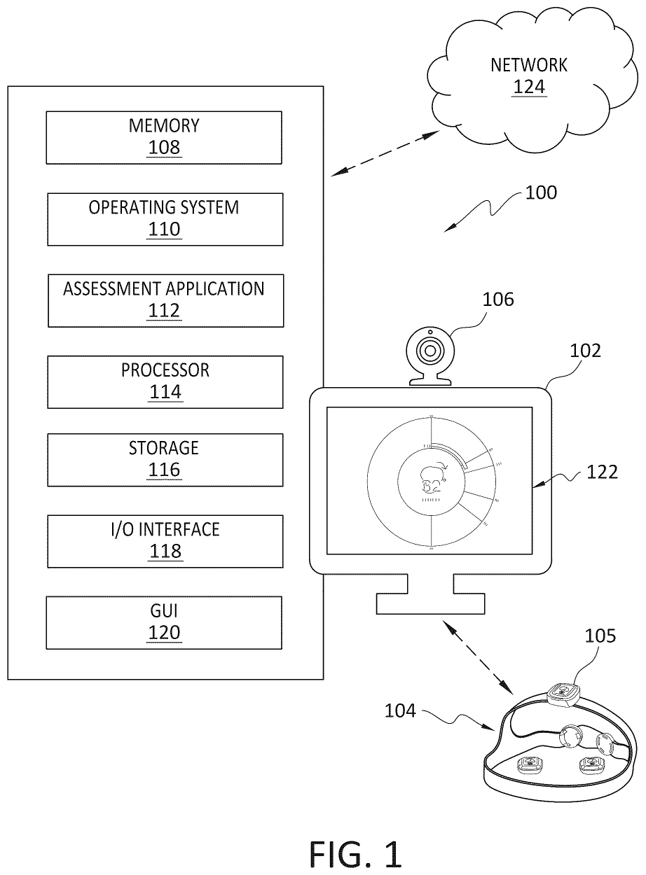

FIG. 1 illustrates a schematic diagram of an assessment system 100 for evaluating individuals with proprioceptive, mobility, and sensorimotor impairments. The system 100 comprises a computing unit 102 having the disclosed assessment application 112, a tracking device 104, and secure cloud-based network 124. The assessment application 112 of the system 100 uses data input received from the tracking device 104 to generate a performance dataset based on a one or more assessments and/or activities. Various movement data is received from the tracking device 104 and subsequently stored and processed with the computing unit 102 during and after the data capture process. Advantageously, clinicians using the system 100 may remotely track an individual's progress, obviating the reliance on self-reporting to confirm patient compliance.

In an embodiment, the tracking device 104 is a distinct hardware component of the system 100. The tracking device 104 may be a wearable device attached to the individual. For example, the tracking device 104 comprises a sensor unit or sensor 105 having one or more inertial measurement units (IMUs) (or similar) attached to an individual. In an embodiment, the tracking device 104 has an adjustable headgear that measures head movement via a Bluetooth-enabled, custom-built sensor 105 that syncs with a computing unit 102 and/or application 112. The tracking device 104 advantageously features a minimalist design that allows a patient to easily progress through physical therapy without excess weight or resistance aggravating their impairment. In an embodiment, the tracking device 104 weighs less than 55 g, preferably less than 100 g.

In an embodiment, each sensor 105 contains one or more IMUs that report changes in head angular position by using the IMUs built-in accelerometers and gyroscopes and algorithm. In an embodiment, the tracking device 104 comprises a first sensor 105 attachable to a head, neck, or limb of the human subject and a second sensor (i.e., similar to sensor 105) attachable to a torso or a trunk of the human subject. IMUs of the sensor 105 detect movement in each of the three cardinal axes and can precisely measure three-dimensional rotational velocity, linear acceleration, and magnetic field.

The assessment application 112 utilizes a sensor fusion algorithm to derive the resultant rotational position (orientation) and reduce drift. In an exemplary embodiment, the sensor fusion algorithm is implemented via remote processor (e.g., processor 114) which may be embedded in the sensor 105 of the tracking device 104 before communicating the derived data to the assessment application 112 for further processing by a processor 114 on the computing unit 102. Using Bluetooth, the measurements are wirelessly transmitted to a computing unit 102 and, via the assessment application 112, displayed on a monitor or display 122 the head position and movement in real time. The wireless connection and transmission capability make the system 100 an ideal solution to support patients when practicing their physical therapy activities in the clinic or at home.

The assessment application 112 processes the measurements from the tracking device 104 and produces quantitative 1D, 2D, and/or 3D metrics on head-neck movements i.e., performance datasets. By comparing the data received from the tracking device 104, it is possible to compare individual results and identify healthy and unhealthy musculoskeletal movements to generate a report for evaluating a condition affecting musculoskeletal health of a user. In an embodiment, the report includes a consolidated health index score for a user 101.

The technology of the assessment application 112 can be described as a cloud-based Internet of Healthcare Things (IOHT) application connected to a wired or non-wired device. The wired or wireless recording devices may include the following: computerized tomography (CT-Scan); computers of various sizes and operational capabilities; high-energy electromagnetic radiation X-ray; inertial measurement unit (IMU); IT tablets of all shape, sizes and computing power; microwave diathermy; mobile phones; smart watches; optical sensors; radar operated devices using radio waves to determine the distance angle and radial velocity of individuals; thermal imaging; and video cameras or devices that are capable of recording musculoskeletal movements of individuals.

The computing unit 102 includes a memory 108, operating system 110, assessment application 112, one or more processor(s) 114, storage 116, input-output interface 118, graphical user interface 120, and display 122. The computing unit 102 may represent any suitable type of computer, computing system, server, disk array, or programmable devices such as a handheld device, a networked device, or an embedded device, etc. The computing unit 102 may be in communication with one or more networked computers via one or more networks 124, such as a cluster or other distributed computing system, through the I/O interface 118. The I/O interface 118 is configured to transmit data between the computing unit 102 and the tracking device 104 e.g., via wired or wireless connection.

The processor 114 may include one or more devices selected from processors, micro-controllers, digital signal processors, microcomputers, central processing units, field programmable gate arrays, programmable logic devices (i.e., digital logic circuitry), state machines, logic circuits, analog circuits, digital circuits, or any other devices that manipulate signals (analog or digital) based on operational instructions that are stored in the memory 108. Memory 108 may be a single memory device or a plurality of memory devices including but not limited to read-only memory (ROM), random access memory (RAM), volatile memory, non-volatile memory, static random access memory (SRAM), dynamic random access memory (DRAM), flash memory, or cache memory. Memory 108 may also include a mass storage device such as a hard drive, optical drive, tape drive, non-volatile solid state device, or any other device capable of storing digital information.

The processor 114 may operate under the control of an operating system 110 that resides in memory 108. The operating system 110 may manage computer resources so that computer program code embodied as one or more computer programs, such as the assessment application 112 communicatively connected to memory 108 may have instructions executed by the processor 114. In an alternative embodiment, the processor 114 may execute the assessment application 112 directly, in which case the operating system 110 may be omitted.

The computer storage 116 typically includes at least one hard disk drive and may be located externally to the computing unit 102, such as in a separate enclosure or in one or more networked computers, one or more networked storage devices (including, for example, a tape or optical drive), and/or one or more other networked devices (including, for example, a server). The storage 116 may also host one or more databases communicatively connected to data collected and received in association with the assessment application 112, wherein the storage 116 includes a cloud-based secured HIPAA-compliant database. The storage 116 may be included in the computing unit 102 and/or in a cloud-based network 124 (or service 126).

The graphical user interface 120 may be operatively coupled to the processor 114 of computing unit 102 in a known manner to allow a system operator to interact directly with the computing unit 102. The graphical user interface 120 may include or be communicatively connected to output devices (e.g., display 122) such as video and/or alphanumeric displays, a touch screen, a speaker, and any other suitable audio and visual indicators capable of providing information to the system operator. The graphical user interface 120 may also include input devices (e.g., tracking device 104) and controls such as an alphanumeric keyboard, a pointing device, keypads, pushbuttons, control knobs, microphones, etc., capable of accepting commands or input from the operator and transmitting the entered input to the processor 114.

In a preferred embodiment, the computing unit 102 is communicatively connected to an integrated tracking device 106, e.g., camera, for observing movement and positional performance of a user 101 and/or tracking device 104. The tracking device 106 may be directly and operatively connected to the computing unit 102 and arranged to monitor the user 101 without being directly attached to the individual.

Those skilled in the art will recognize that the computing environment illustrated in FIG. 1 is not intended to limit the solution(s) in the present disclosure. In addition, various program code described herein may be identified based upon the application or software component within which it is implemented in a specific embodiment of the invention. However, it should be appreciated that any particular program or hardware nomenclature that follows is used merely for convenience, and thus the disclosed system and method should not be limited to use solely in any specific application identified and/or implied by such nomenclature.

It should be further appreciated that the various features, applications, and devices disclosed herein may also be used alone or in any combination. Moreover, given the typically endless number of ways in which computer programs may be organized into routines, procedures, methods, modules, objects, and the like, as well as the various ways in which program functionality may be allocated among various software layers that are resident within a typical computing system (e.g., operating systems, libraries, APIs, applications, applets, etc.), and/or across one or more hardware platforms, it should be appreciated that the invention is not limited to the specific organization and allocation of program or hardware functionality described herein.

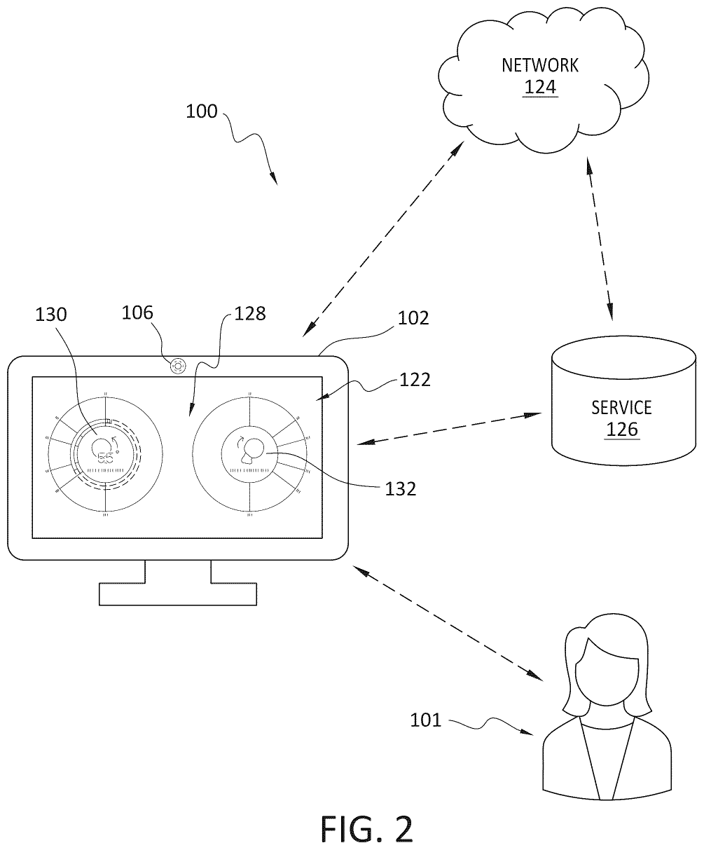

FIG. 2 illustrates the system 100 further including a service 126 being communicatively coupled to both the computing unit 102 and network 124. In some cases, the service 126 can be a deterministic service that operates fully given a set of inputs and without a randomization factor. In other cases, service 126 can be or can include a machine learning (ML) or artificial intelligence engine to enable the service 126 to operate even when faced with a randomization factor. In some implementations, the service 126 is a cloud service operating in a cloud environment. In some implementations, the service 126 is a local service operating on a local device. In some implementations, the service 126 is a hybrid service that includes a cloud component operating in the cloud and a local component operating on a local device. These two components can communicate with one another.

The service 126 is generally tasked with operatively connecting the computing unit 102 with the network 124 or another computer. In an embodiment, the service 126 functions as a proxy server or intermediary between the computing unit 102 and the network 124. In an alternative embodiment, the service 126 functions as a virtual machine. The service 126 may receive incoming data, i.e., a software update, from a network 124. The service 126 may be configured as a collective database storing (e.g., via storage 116) previously obtained data (e.g., historical performance datasets) based on performance datasets of the individuals and/or normative data of a representative population. The historical performance dataset is preferably collected from a group of individuals (population). Historical performance data from the respective individual may also be used in the dynamic calculation together with a population historical dataset. Additionally, patient characteristics, including symptoms, might be factored into the dynamic calculation as well, including parameters such as age, gender, weight etc.

FIG. 2 illustrates the general architecture and assessment setup for the system 100. The tracking device 106, e.g., camera or motion capture device, is integrated with the computing unit 102 and arranged to monitor and record a user 101 during execution of various assessments and activities of the assessment application 112. The computing unit 102 collects movement data from the user 101 (e.g. via tracking device 104) during the assessments and activities.

FIG. 2 further illustrates an instructional interface 128 as part of the assessment application 112. During an initial period of using the instructional interface 128, a user 101 may select which assessment to perform. The instructional interface 128 may prompt the user to select at least one assessment, including Butterfly, joint position error, and range of motion assessments. Non-limiting examples of the computer-directed assessment include the Butterfly test (inclusive of variations of a visible path, small/large path, and non-centered path), range of motion assessment, joint position error test with and without offset, controlled articular rotations (CARs), set points in range (start in center and then reach targets at different locations with the cursor), zigzag test and similar (trace a visible path with the cursor at a self-selected speed), smooth pursuit neck torsion test, gaze stability (e.g., maintaining gaze while moving the head), saccadic eye movements, and discrete eye-head coordination. The assessment may also refer to rehabilitation exercise and training tasks for rehabilitative and performance enhancement or instructional purposes, respectively.

In an embodiment, the at least one assessment is directed to evaluating at least one of range of motion, proprioception, balance, neuromuscular control, sensorimotor control, oculomotor control, pupillary response, sensorimotor integration, eye-head-neck coordination, vestibular function, cognition, and strength. The instructional interface 128 may further prompt the user 101 to select or record background information and symptoms that apply prior to performing the assessments. Symptoms may include physical, emotional, cognitive and sleep related symptoms such as pain intensity, anxiety, concentration problems, and sleep disturbance. Other symptoms can include headache, dizziness, balance problems, pain frequency, muscle stiffness, muscle tenderness, radiating pain, depression, memory problems, brain fog, blurred vision, sensitivity to light, fatigue, drowsiness, and nausea.

In an embodiment, the instructional interface 128 is configured to prompt the user 101 to complete a symptoms assessment by recording or ranking a number of predetermined symptoms. The symptoms could be ranked using a numerical scale, a descriptive/worded scale, a slidable GUI, a pictorial scale, a colored scale, and/or the like. The user can also input “custom symptom” which are not included in a predetermined list of symptoms provided by the instructional interface. Additionally, there can also be custom entry field to add a binary or “yes/no” type of entry, e.g. in the category for “History related to current symptoms.” Regarding the duration of the symptom of interest, the user 101 can be prompted to mark the symptom as being acute or subacute, or chronic. The user 101 can further be prompted to label history related to the current symptom as including head impacts or concussions, idiopathic neck pain, traumatic neck pain, and/or degenerative neck pain.

In an embodiment, the user 101 can be prompted to rate the following symptoms: physical symptoms (e.g., balance problems, dizziness, headache, jaw pain or dysfunction, neck pain, visual disturbances), emotional symptoms (e.g., anxiety, depression), cognitive symptoms (e.g., difficulty, concentrating, difficulty remembering, feeling like in a fog), and sleep symptoms (e.g., sleep disturbances, insomnia, sleep apnea).

In an embodiment, the instructional interface 128 further prompts the user 101 to select at least one activity to complete while performing the at least one assessment. For example, when the at least one assessment includes a range of motion assessment, the one or more activities may include extension, flexion, left-right rotation, and left-right lateral flexion. In an embodiment, the at least one activity includes at least one of flexion, extension, rotation, abduction, adduction, circumduction, elevation, depression, pronation, supination, protraction, and retraction using a body portion of the user 101. Other activities can include body sway to measure the extent of center of gravity excursions, eye movement to measure oculomotor capabilities, and pupil dilation to assess pupillary response. For example, for assessment that include balance evaluation, the at least one activity could include a static balance test (e.g., standing with feet together, a tandem stance, or a single-leg stance) and a dynamic balance test (e.g., heel-to-toe walk, sidestepping, stair climbing, or walking with knee lifts). For assessments that include evaluating a pupillary response or ocular movement, the at least one activity could include a direct light reflex, a consensual light reflex, an accommodation reflex, and eye movements performed by one or more eyes of the user. The instructional interface 128 may visually list or audibly convey instructions specific to the at least one assessment. Instructions may include environmental arrangement, wearable device setup, and directions for the user and/or provider to complete. In an embodiment, the instructional interface 128 prompts the user 101 to select a set number of repetitions for the activities of the assessment.

In an embodiment, calibration for the at least one assessment occurs by connecting the tracking device 104 to the computing unit 102. The instructional interface 128 may prompt the user 101 to place the tracking device 104 on a substantially flat or horizontal surface for a predetermined period of time. In an alternative embodiment, wherein an integrated tracking device 106 is used for observing movement and positional performance of the user 101, motion capture calibration is performed before beginning the at least one assessment. Calibration may occur in the background of the assessment application 112, or the instructional interface 128 may prompt the user 101 to perform steps to complete the calibration. The calibration may be dynamic, wherein time allowance for calibrating the tracking device 106 varies between assessments and/or users. The calibration may also be device specific and dependent on the type of tracking device 106 used.

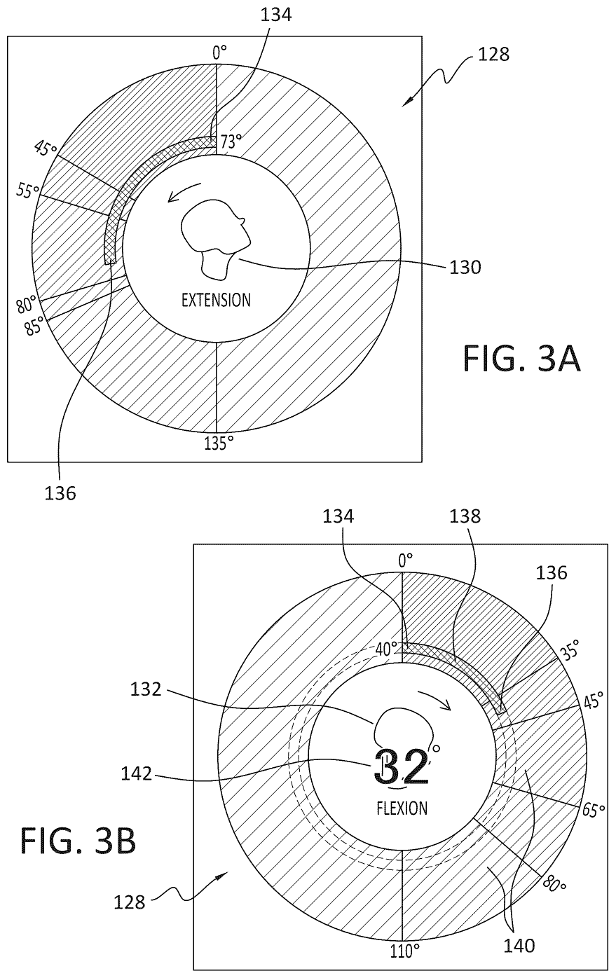

FIG. 3A illustrates the instructional interface 128 displaying a first instructional image 130 of an activity (e.g., extension) to perform during an assessment (e.g., range of motion). One skilled in the art will recognize that features of one instructional image can relate to another instructional image described in the present disclosure. As depicted, the activity relates to extension of the neck and the instructional image 130 includes a graphical representation of the activity. In an embodiment, the instructional image 130 is static or motionless in appearance and may include instructional arrows to portray actions or steps that the user is to complete during the activity. In an alternative embodiment, the instructional image 130 is in motion or moving in appearance to portray actions or steps that the user is to complete during the activity.

The assessment application 112 of the system 100 is configured to automatically determine a starting point 134 and endpoint 136 for each activity. The starting point 134 and endpoint 136 may be marked or recorded on the instructional interface 128. The assessment application 112 defines the starting point 134 and endpoint 136 using kinematic data corresponding to the user 101. The assessment application 112 obtains the kinematic data, e.g., using one or more tracking devices 104, 106, and detects movement in the anatomical body planes. The kinematic data is defined by the anatomical body planes, including coronal (frontal), sagittal (longitudinal), and transverse (axial) planes.

The assessment application 112 automatically determines when the user 101 has completed an activity, e.g., completing a circular path, and subsequently calculates the area surrounded or between points of interest, such as the starting point 134 and endpoint 136. In an embodiment, the assessment application 112 calculates an assumption of connecting points. The assessment application 112 can detect if the user decides to change movement direction and/or perform another test (e.g., used in a CARs assessment). The assessment application 112 can incorporate one or more audible indicators (i.e., sounds) to indicate user movement and state of the assessment and/or activity. For example, the sounds can be incorporated in activities associated with joint position error to assist the user 101 perform the assessment and/or exercise.

FIG. 3B illustrates the instructional interface 128 displaying a second instructional image 132 of another activity (e.g., flexion) to perform during the assessment (e.g., range of motion). The instructional interface 128 includes at least one performance indicator 138 to display, in real time, the quantified level execution of the activity while the user 101 completes the activity. In an embodiment, the performance indicator 138 is graphically portrayed between the starting point 134 and endpoint 136 of the activity, i.e., the performance indicator 138 begins at the starting point 134 and follows a course toward the endpoint 136. The instructional interface 128 may include a numerical index 142 to display a quantified metric or measurement relating to the user's 101 performance during the activity. The numerical index 142 can also be broadcast to the user using audio, e.g., produced from a speaker connected to the computing unit 102.

In an embodiment, the instructional interface 128 includes a target range model 140 to visually indicate a predetermined range of desired (or optimal) performance of the activity (e.g., in degrees). The target range model 140 can be based on individual user characteristics, a comparison population (i.e., healthy population, patient population, or another population comprising historical performance data with the instructional interface), and/or another sampled statistic. Exemplary user characteristics that may be factored into the generation of the target model 140 can include age, gender, weight, symptoms, and the like.

Advantageously, because the assessment application 112 obtains the kinematic data, e.g., using one or more tracking devices 104, 106, and detects movement in the anatomical body planes, the instructional interface 128 is arranged to automatically determine which activity (e.g., flexion or extension) the user 101 begins to perform.

Based on the user 101 input of the kinematic data and registered movement, the instructional interface 128 automatically updates the instructional image 130, 132 to correspond with the appropriate activity that the user 101 is actively completing. The assessment application 112 can further detect whether the same activity is being completed in sequence (e.g., repeating the activity of extension), or whether the user 101 is switching between activities (e.g., toggling between flexion and extension). Likewise, the corresponding starting points, endpoints, performance indicators and numerical indices are updated to correspond to the activity of interest (i.e., actively being performed by the user 101).

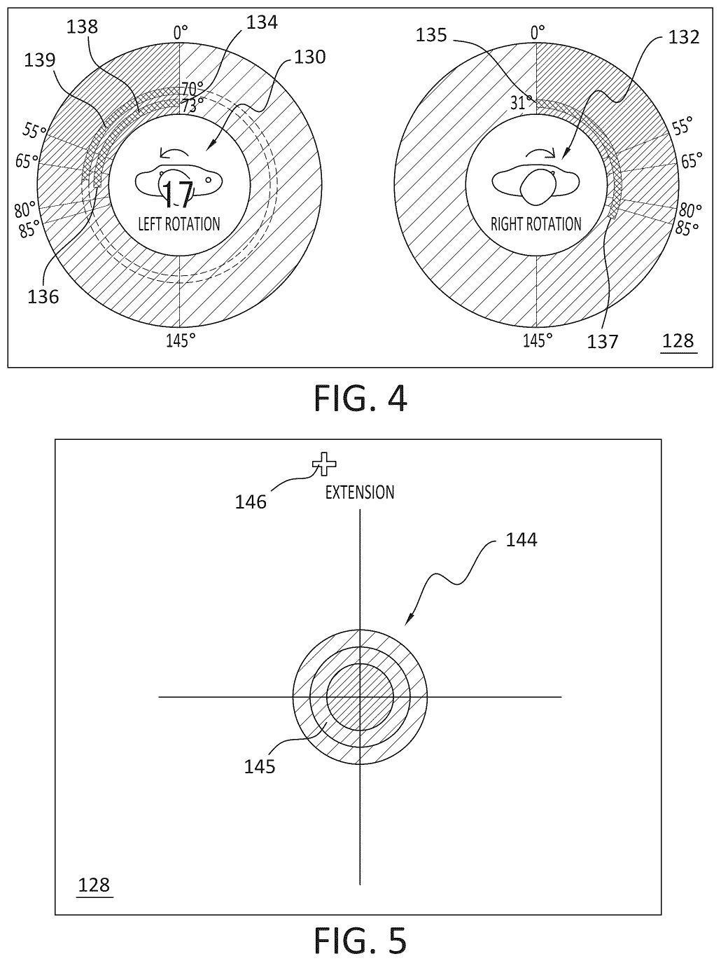

FIG. 4 illustrates an embodiment of the instructional interface 128 displaying both first and second activities with first and second instructional images 130, 132. The instructional interface 128 is configured to foreground (emphasize or highlight) the instructional image 130 that corresponds to the activity that is actively being completed by the user. As depicted in FIG. 4, the first instructional image 130 is foregrounded, while the second instructional image 132 is muted or made less noticeable so as to not confuse the user 101 during completion of the at least one assessment. In an embodiment, the first instructional image 130 is replaced by the second instructional image 132 after the second activity is detected as being performed by the user 101.

Multiple performance indicators 138, 139 can be displayed for each activity, including a past performance indicator 138 that remains on display while a real-time performance indicator 139 is generated during completion of a subsequent activity. Such toggling ability between activities (and, thus, instructional images) is automatically detected by the assessment application 112 and performed by the instructional interface 128 to provide an automated, accurate experience without input from a human operator to guide each activity. Additionally, each activity includes a starting point 134, 135, and an endpoint 136, 137, which can be visibly denoted by performance indicators, and the starting point of the assessment corresponds to or is the same as the starting point of the first activity, wherein the endpoint of the assessment corresponds to or is the same the endpoint of the last activity.

The assessment application 112 determines when the kinematic data falls within a predetermined target range for a starting point (e.g., starting point 134), tracks the movement of the user 101 and corresponding kinematic data (e.g., using at least one tracking device 104, 106), and also determines when the kinematic data falls within a predetermined target trance for an endpoint (e.g., endpoint 136) of the activity. The at least one assessment includes a starting point for each activity and an endpoint for each activity, wherein the first starting point for the first activity corresponds to a general starting point for the at least one assessment and the last endpoint for the last activity corresponds to a general endpoint for the at least one assessment.

FIG. 5 illustrates an embodiment of the instructional interface 128 arranged to display an instructional image 144 corresponding to a joint position error assessment, i.e., a clinical assessment for proprioception. The instructional image 144 may include a positional target 145 and moveable cursor 146. The instructional interface 128 may include visible and/or hidden graphics (e.g., positional target 145 and cursor 146) from the individual 101 during execution of one or more activities and/or assessments. In an embodiment, the joint position error assessment defines an error in repositioning the head to a neutral head position (NHP) or to a target head position (THP), which is offset from the NHP, using an audible cue from the assessment application 112.

Advantageously, the assessment application 112 may simultaneously or concurrently complete multiple assessments. In an embodiment, a first assessment is provided by the assessment application 112 for evaluating the condition and obtaining one or more first evaluations of range of motion, proprioception, balance, neuromuscular control, sensorimotor control, oculomotor control, pupillary response, sensorimotor integration, eye-head-neck coordination, vestibular function, cognition, and strength. Additionally, a second assessment is provided by the assessment application 112 for evaluating the condition and obtaining one or more second evaluations of range of motion, proprioception, balance, neuromuscular control, sensorimotor control, oculomotor control, pupillary response, sensorimotor integration, eye-head-neck coordination, vestibular function, cognition, and strength. In other words, the first assessment and the second assessment include at least one concurrent activity to obtain the first and the second evaluations

To save time and consolidate testing, the simultaneous execution for both first and second assessments provide insight regarding the condition based on different evaluations. In other words, the one or more first evaluations are different from the one or more second evaluations. For example, both range of motion and joint position error assessments may be conducted at the same time. Because the assessment application 112 automatically determines starting points and endpoints, said starting points and end points may be used for assessing both proprioception and range of motion. In an embodiment, the assessment application 112 further automatically determines points of interest between starting points and end points. For example, when performing a combined assessment of range of motion and joint position error, the maximum range of motion value would occur between the starting and end points (i.e., a point of interest) because the starting point is defined as the neutral head position and the ending point is defined as the perceived neutral head position. Such provides a clear distinction as the main metric of the joint position error test, the difference between the starting neutral position and the patient's perceived neutral position after an active movement.

While the joint position error assessment assesses cervicocephalic proprioception ability of the user 101 (i.e., accuracy of the user 101 in returning to a starting point or neutral position), the range of motion assessment assess maximum movement ability of the user (i.e., maximum flexion, extension, and rotation metrics). Completing both assessments simultaneously saves time and storage costs otherwise required for completing both assessments separately. Similarly, in an alternative embodiment, the assessment application 112 is arranged to complete a Butterfly test with an eye tracking test, thereby evaluating movement of the head and eyes. Another combination of assessments includes using the Butterfly test with pupillometry to measure pupil response as a proxy for attention and/or cognitive load.

In such embodiments, where the Butterfly test is combined with an ocular test, eye-movement abnormalities may be observed in relative head and neck movements. The assessment application 112 can objectively quantify the eye-movements, in combination with Butterfly readings, such as time-on-target and overshoots/undershoots, to aid the diagnosis of musculoskeletal or neurological conditions of a user. Reports generated by the assessment application 112 can progressively monitor the correlation of eye-tracking with a user's physical performance of the Butterfly test.

A controlled articular rotation assessment may also be completed with a standard (i.e. uniplanar) range of motion assessment. In such an embodiment, the assessment application 112 detects when the user begins the assessment, when the user has moved to a maximum position of displacement, when the user begins articular rotation, when the user has circled, whether the user decides to change direction, and when the assessment is complete.

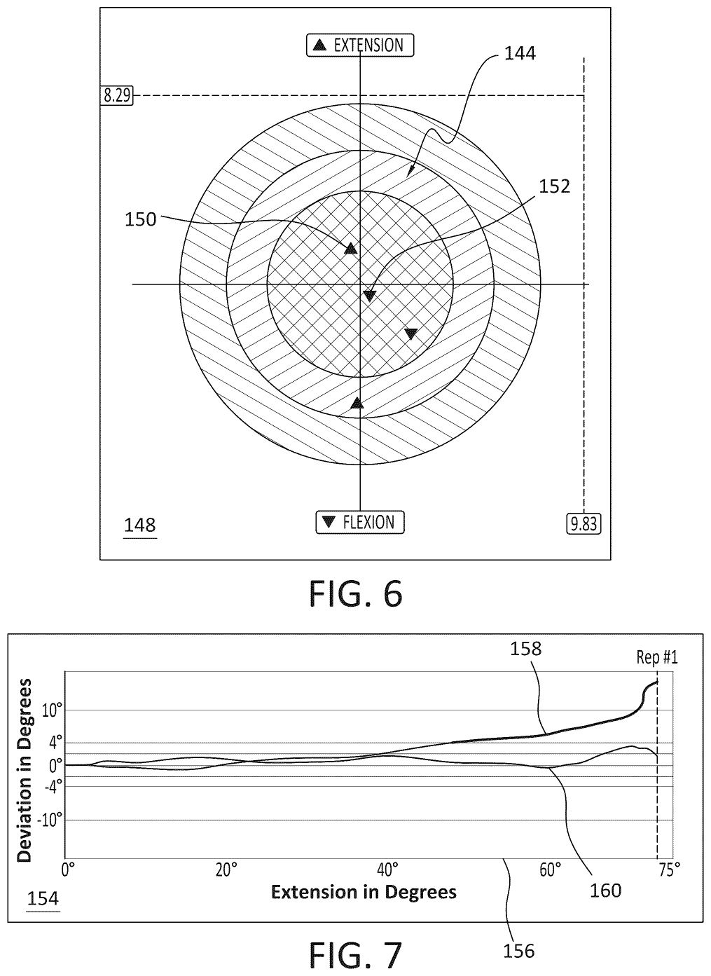

FIG. 6 illustrates a graphical scorecard 148 for the at least one assessment relating to proprioception. The graphical scorecard 148 includes the instructional image 144 from the relevant activity and/or assessment, including grading icons 150, 152 to show performance marks of the user 101. Each grading icon 150, 152 may relate to a specific activity (e.g., extension or flexion), wherein the first grading icon 150, which relates to extension, is different (i.e., a different shape or icon) from the second grading icon 152, which relates to flexion. The graphical scorecard may be provided in a report, generated after completion of one or more activities and/or assessments, from the assessment application 112.

Advantageously, the graphical scorecard 148 of the instructional interface 128 can be configured to zoom in towards a section of interest on the instructional image 144, and may also be configured to zoom out to include or encompass all icons or points of interest. In an embodiment, the assessment application 112 records positional data of the cursor 146 beyond the physical boundary displayed on the screen. In other words, if the cursor 146 goes off screen (i.e., beyond boundaries of the physical display 122) during an activity, the assessment application 112 continuously records positional data so that, following completion of the at least one assessment, the instructional interface 128 is arranged to allow zooming out with the scorecard to comprehensively include the icons or points of interest.

FIG. 7 illustrates a graphical representation 154 of plane association. As noted above, the assessment application 112 obtains the kinematic data, and detects movement in the anatomical body planes, including coronal (frontal), sagittal (longitudinal), and transverse (axial) planes. The graphical representation 154 illustrates the relationship between planes, wherein the y-axis relates to a recorded metric in a first plane 156 (e.g., extension in a first plane in terms of degrees) and the x-axis relates to deviation from the first plane 156 into the second plane 158 and third plane 160. For example, the first plane 156 may correspond to the sagittal plane in which the user 101 performs the activity of extension. From the starting point (i.e., zero degrees of extension) to the endpoint (i.e., maximum degree of extension), the assessment application 112 records the level of deviation from the sagittal plane into the coronal and transverse planes. In an embodiment, greater levels of deviation are denoted by color change, and certain colors or levels of deviation may generate alerts of abnormality or concern.

The plane association can be displayed to the user 101 during completion of an assessment and/or following completion of the assessment. Feedback (e.g., in the form of a visual or audible indicator) may be provided to the user 101 to indicate proper alignment within or deviation from a specific plane in which the user is performing an activity. For example, during completion of a neck extension activity, if the user 101 deviates from the sagittal plane by a predetermined amount that is considered to be notable or significant, the instructional interface 128 may indicate to the user 101, using an audible or visual alert such as a color or icon change, that the user 101 is off course and further prompt the user 101 to physically course-correct the action during completion of the activity.

In an embodiment, an audible indicator is provided for the joint position error assessment. The audible indicator is used to help the patient go back to a “center” or neutral starting position after relocation without having to open the eyes. The audible indicator can include continuous dynamic audio that changes as the patient moves and is constructed in a way that guides the patient to move in the appropriate direction and distance to return to the starting position. Traditionally, a clinician needs to manually move the patient's head back to the center after the active movement and relocation attempt. The proposed embodiment provides a solution to automatically move from the relocation point to the starting position. Examples of the dynamic audio changes can relate to increasing and/or decreasing volume, pitch, and/or tone.

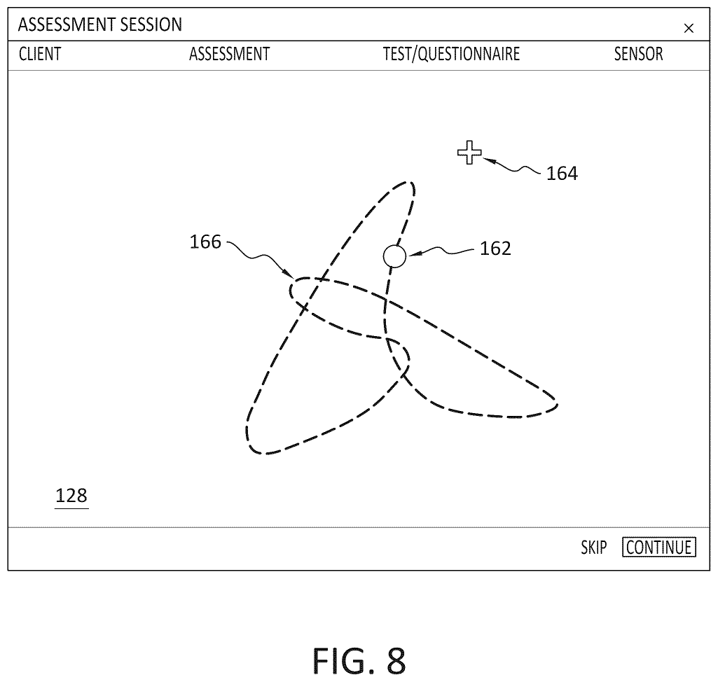

FIG. 8 illustrates the instructional interface 128 configured to execute a Butterfly test for the user 101. The activity of the assessment is to follow a moving indicator 162, using a cursor 164, along a computer generated path 166. In an embodiment, the instructional interface 128 prompts the user 101 to follow the moving indicator 162 along the computer-generated path 166 with the cursor 164 by moving the head, neck, or limb of the subject. The computer-generated path 166 may be presented on the display 122 either as a visible path, or hidden from the view of the user while the moving indicator 162 follows the invisible computer-generated path 166. In an embodiment, the assessment application 112 records performance data based on a trajectory path of the cursor 164, and a correlation and/or deviation between the trajectory path of the cursor 164 and the computer-generated path 166. The correlation and/or deviation can further be analyzed using movement parameters (metrics).

The assessment application 112 can provide an indicator to alert the user 101 when the cursor 164 has deviated from the computer generated path 166 by a predetermined amount or distance. The indicator may be provided in the form of an audible alert, visible change on the display 122 (i.e., color changing cursor 164, changing an icon of the cursor 164 (e.g., from a default icon to an icon signifying that the cursor 164 has deviated from the computer generated path 166 by a notable amount), changing a color scheme shown on the entire display 122), or combinations of both. Such indicators may be provided in Butterfly tests with and without the moving indicator 162.

In an embodiment, the moving indicator 162 changes color based on the tracking performance of the user 101 using the cursor 164. For example, when the user 101 remains on target, or is otherwise not over- or under-shooting the mark, the moving indicator 162 is green. When the user 101 deviates from the computer-generated path 166 by a predetermined about, however, the moving indicator 162 changes to a red color. Additional colors associated with the level of deviation are also contemplated. Thus, the instructional interface 128 provides real-time feedback to the user 101 to indicate performance accuracy and/or prompt course correction from the user 101.

In an embodiment, the Butterfly test involves a variable speed along the computer-generated path. The Butterfly test may include variable pattern difficulty (e.g., easy, medium, or difficult). In an embodiment, the activity of the assessment for the Butterfly test includes tracing the computer generated path 166, using the cursor 164, without the moving indicator 162.

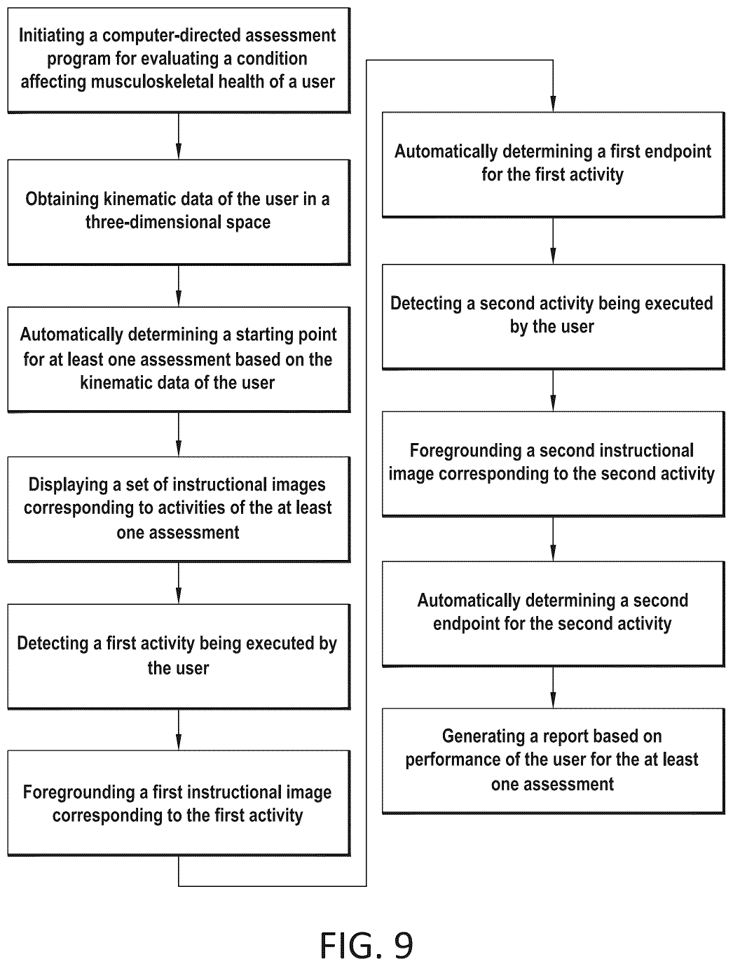

FIGS. 9-12 illustrate exemplary flowcharts of methods implemented by the disclosed system 100. One skilled in the art understands that certain steps in one method can apply to another method. FIG. 9 illustrates a flowchart for a method implemented by the disclosed system 100. In an embodiment, the method for implementing the computer-directed assessment program using an instructional interface with a set of instructional images includes the following steps: initiating a computer-directed assessment program for evaluating a condition affecting musculoskeletal health of a user; obtaining kinematic data of the user in a three-dimensional space; automatically determining a starting point for at least one assessment based on the kinematic data of the user; displaying a set of instructional images corresponding to activities of the at least one assessment; detecting a first activity being executed the user; foregrounding a first instructional image corresponding to the first activity; automatically determining a first endpoint for the first activity; detecting a second activity being executed by the user; foregrounding a second instructional image corresponding to the second activity; automatically determining a second endpoint for the second activity; and generating a report based on performance of the user for the at least one assessment. Regarding initiation of the computer-directed assessment, an embodiment can include selection, by a user (e.g., clinician), of which assessment to initiate, followed by producing a prompt (e.g., GUI or keyboard button click) within the instructional interface of the application for the user to select once ready to begin the activity.

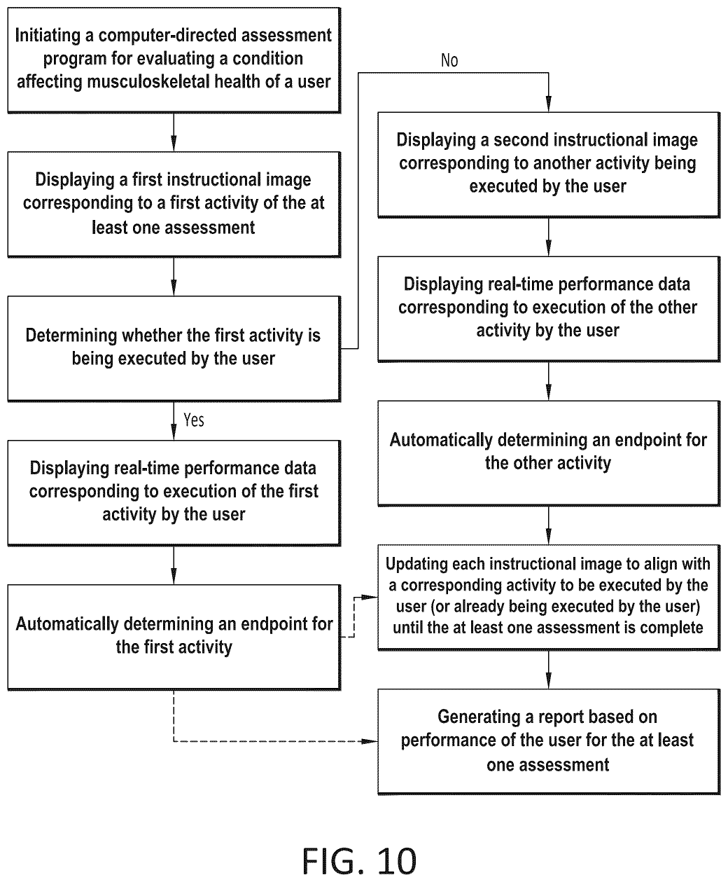

FIG. 10 illustrates a flowchart of a method for implementing the computer-directed assessment program including automatically updating instructional images to align with corresponding computer-prompted activities. The method includes the following steps: initiating a computer-directed assessment program for evaluating a condition affecting musculoskeletal health of a user; displaying a first instructional image corresponding to a first activity of the at least one assessment; and determining whether the first activity is being executed by the user. If the computer-directed assessment program determines that the first activity is actually being completed by the user, the method further includes the following steps: displaying real-time performance data corresponding to execution of the first activity by the user; automatically determining an endpoint for the first activity; updating each instructional image to align with a corresponding activity to be executed by the user (or already being executed by the user, e.g., with a joint position error assessment, wherein such occurs before automatically determining an endpoint of the related activity) until the at least one assessment is complete; and generating a report based on performance of the user for the at least one assessment. Alternatively, in embodiments where the at least one assessment includes a single activity, the system can generate a report following automatically determining an endpoint for the single activity.

If the computer-directed assessment program determines that the first activity is not actually being completed by the user, the method further includes the following steps: displaying a second instructional image corresponding to another activity being executed by the user; displaying real-time performance data corresponding to execution of the other activity by the user; automatically determining an endpoint for the other activity; updating each instructional image to align with a corresponding activity to be executed by the user (or already being executed by the user) until the at least one assessment is complete; and generating a report based on performance of the user for the at least one assessment.

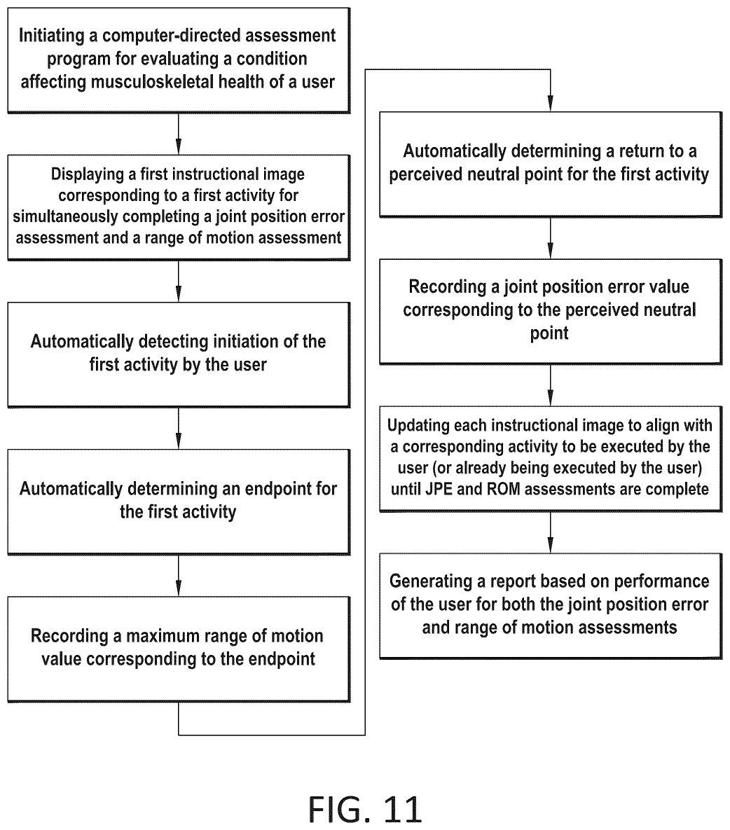

FIG. 11 illustrates a flowchart of a method for implementing the computer-directed assessment program including simultaneously completing a joint position error assessment and a range of motion assessment. The method includes the following steps: initiating a computer-directed assessment program for evaluating a condition affecting musculoskeletal or neuromuscular health of a user; displaying a first instructional image corresponding to a first activity for simultaneously completing a joint position error assessment and a range of motion assessment; automatically detecting initiation of the first activity by the user; automatically determining an endpoint for the first activity; recording a maximum range of motion value corresponding to the endpoint; automatically determining a return to a perceived neutral point for the first activity; recording a joint position error value corresponding to the perceived neutral point; updating each instructional image to align with a corresponding activity to be executed by the user (or already being executed by the user) until joint position error and range of motion assessments are complete; and generating a report based on performance of the user for both the joint position error and range of motion assessments.

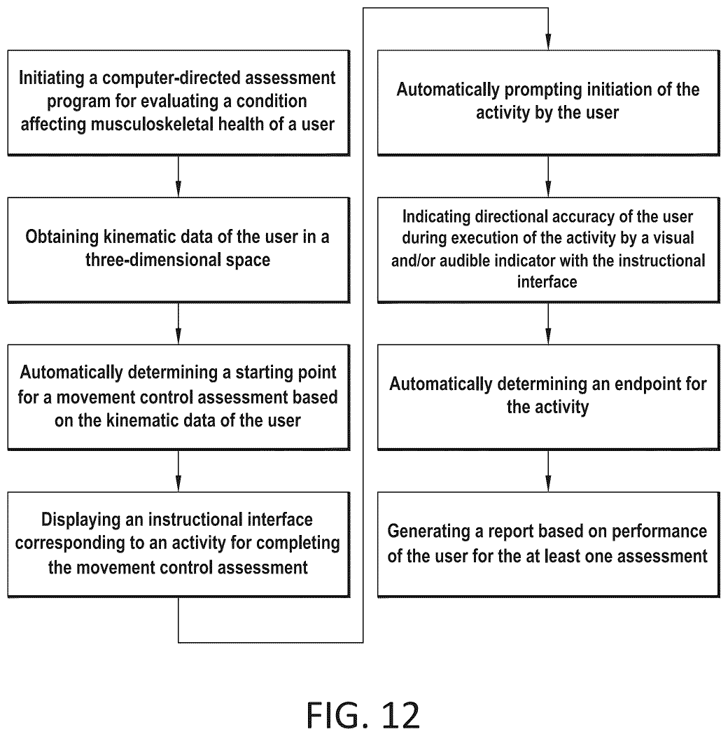

FIG. 12 illustrates a flowchart of a method for implementing the computer-directed assessment program including visually indicating movement accuracy of a user during execution of at least one computer-prompted activity by a change in color with the instructional interface. The method includes the following steps: initiating a computer-directed assessment program for evaluating a condition affecting musculoskeletal health of a user; obtaining kinematic data of the user in a three-dimensional space; automatically determining a starting point for a movement control assessment based on the kinematic data of the user; displaying an instructional interface corresponding to an activity for completing the movement control assessment; automatically prompting initiation of the activity by the user (e.g., in the form of a visual or audible timer or signal); visually indicating movement accuracy of the user during execution of the activity by a change in color with the instructional interface; automatically determining an endpoint for the activity; and generating a report based on performance of the user for the at least one assessment.

### Exemplary Embodiment to Generate and Administer Rehabilitation Exercises and Training Tasks

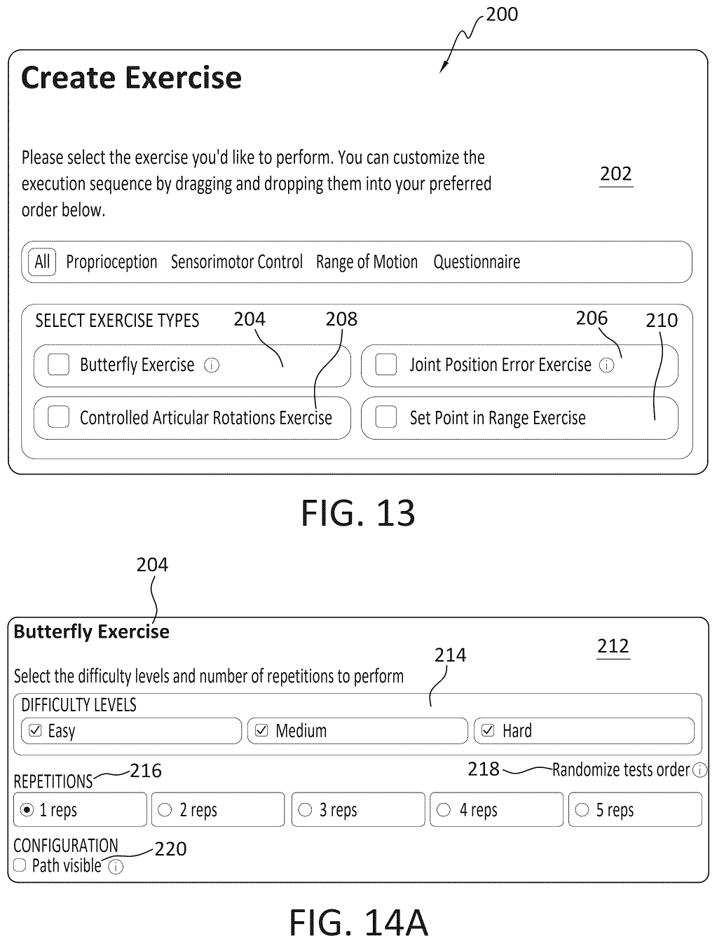

FIGS. 13-17 illustrate exemplary interfaces for an exercise module 200 of the assessment application 112 implemented by the system 100. In FIG. 13, the exercise module 200 is presented on a graphical user interface 202 e.g., on the display 122 of a computing unit 102. The user interface 202 displays one or more exercise types 204, 206, 208, 210 arranged for selection by a user of the application 112. In an embodiment, the exercise types include a Butterfly exercise 204, a joint position error exercise 206, a controlled articular rotation exercise 208, and a set point range exercise 210. One skilled in the art will recognize that other exercises may be provided for selection by the user. Additionally, the exercise module 200 can include computer-directed exercises directed to proprioception, sensorimotor control, range of motion, oculomotor control, balance, strength, and the like. The one or more exercises 204, 206, 208, 210 can be customized in sequence by dragging and dropping them via the user interface 202 into a preferred order.

The exercise module 200 may also include instructions to complete one or more questionnaires directed to assessing and monitoring a condition (e.g., neck pain) and related issues. The questionnaire can be used in combination with the exercise module 200 to collected information about pain levels, activity limitations, and historical details about the condition of the user.

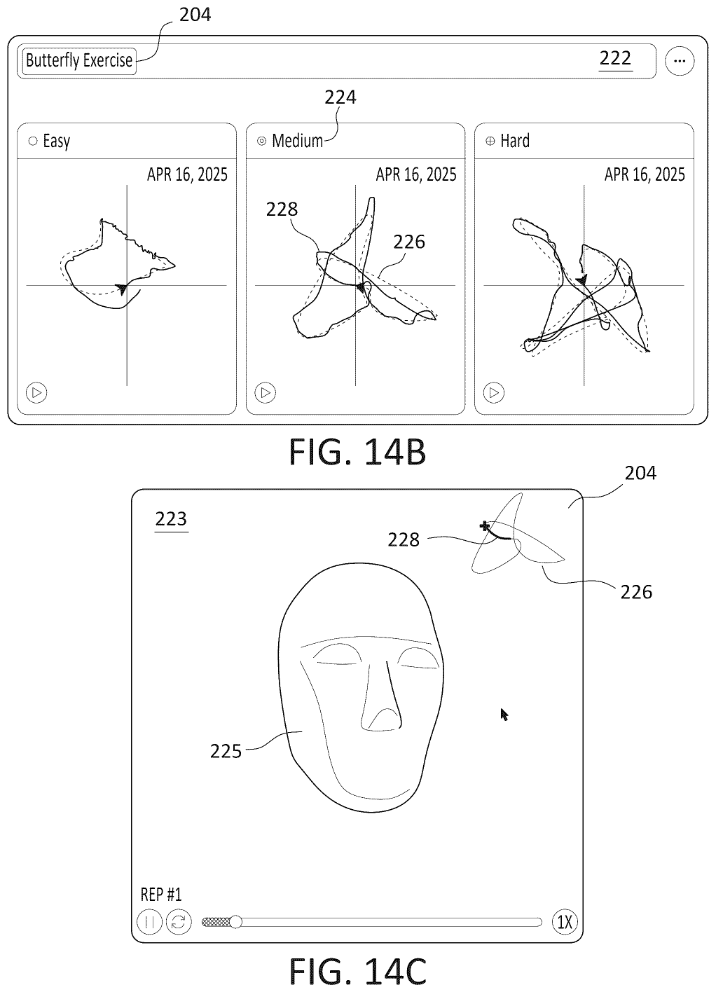

FIG. 14A illustrates an exemplary user interface 212 for the Butterfly exercise 204. The Butterfly exercise 204 is designed to improve sensorimotor control of the cervical spine. During the Butterfly exercise 204, the user attempts to follow a moving target on the display or computer screen as accurately as possible with a cursor e.g., controlled by a head-mounted sensor. The Butterfly exercise 204 can be performed with the butterfly path itself visible or invisible, moving at an unpredictable velocity along a predetermined trajectory. Such a feature can be activated using a configuration icon 220. The user interface 212 can display two or more selectable difficulties 214, such as easy, medium, or hard. In an embodiment, the Butterfly exercise 204 can use a variety of patterns/trajectories for each difficulty level. For example, when 3 repetitions of easy and 5 repetitions of medium are selected, the application 112 of the exercise module 200 randomly selects which 3× easy and 5× medium patterns will be presented to the user. In an embodiment, the patterns for the Butterfly exercise 204 are pre-generated and stored in a database or generated on the fly according to an algorithm that accounts for details and/or historical performance of the user with the assessment 112. The user interface 212 can also include a randomizer 218 to produce and unpredictable or random sequence of the selected difficulties 214. The randomizer 218 can also be implemented with other sequences of different exercises. Additionally, the user interface 212 can display a selectable repetition option 216, which can be arranged to display a set number of selectable repetitions or a customizable field for the user to manually enter a desired number of repetitions of the Butterfly exercise 204. In an embodiment, the user interface 212 includes at least one of the following additional interactive features: an option to increase or decrease the speed of the moving target; an option to increase or decrease the size of the target; an option to increase or decrease the size/amplitude of the trajectory; and an option to offset the position of the trajectory. Such features can also be implemented with other assessment and exercises of the present disclosure.

Before the Butterfly exercise 204 is conducted, the following recommendations can be provided and/or displayed to the user: for proper sitting posture (i) sit in a firm chair with a back support, (ii) feet are to rest on the ground or firm surface, hip distance apart, and (iii) arms resting in lap, hands not touching; and for proper positioning of headgear (i) the sensor is horizontal on top of the user's head, (ii) headgear is firmly tightened without discomfort, and (iii) the headgear is centered along the midline. The user may also be further prompted or instructed by the assessment application 112 to (i) maintain controlled and smooth movements throughout the test, (ii) move the cursor or cross by moving their head, and/or (iii) instruct the user to limit compensation from shoulders and/or the trunk. For example, before, after, or during the observing or receiving of a movement or measurement that falls outside of a predetermined setting (i.e., the movement of the user exceeds an acceleration threshold or falls outside of a range or window), the assessment application 112 can display such prompts or instructions. The same recommendations can be provided for different exercises.

FIG. 14B illustrates an exemplary user interface 222 displaying results of the Butterfly exercise 204. The user interface 222 can present one or more diagrams 224 that illustrates the performance of the user for the Butterfly exercise 204. The number of diagrams 224 is dependent on the number of patterns or trajectory paths completed, e.g., a diagram 224 is generated for each Butterfly exercise, e.g., a user may complete five easy patterns (and no medium or hard patterns), wherein five diagrams are subsequently displayed. Each diagram 224 also displays the predetermined trajectory 226 for the selected difficulties and one or more recorded paths 228 of the user's performance. The diagram 224 can be interactive and arranged to replay the creation or generation of the paths 228 for each selected difficulty 214.

FIG. 14C illustrates a playback user interface 223 for graphically presenting the performance of the user for the Butterfly exercise 204. Like the diagrams 224 for displaying the results of the Butterfly exercise 204, the playback user interface 223 shows the predetermined trajectory 226 the recorded path 228 of the user's performance. The playback user interface 223 is also provided with a 3D model 225 of the user's performance to represent a visualization of the 3D movement of the user's head (or another body part) during the Butterfly exercise 204. One skilled in the art will recognize that the 3D model 225 can be applied to other exercises and assessments of the present disclosure. In an embodiment, the 3D model is synchronized with the recorded path 228 of the user's performance, wherein the 3D model depicts 3D movement and the recorded path 228 depicts 2D movement for the exercise and/or assessment.

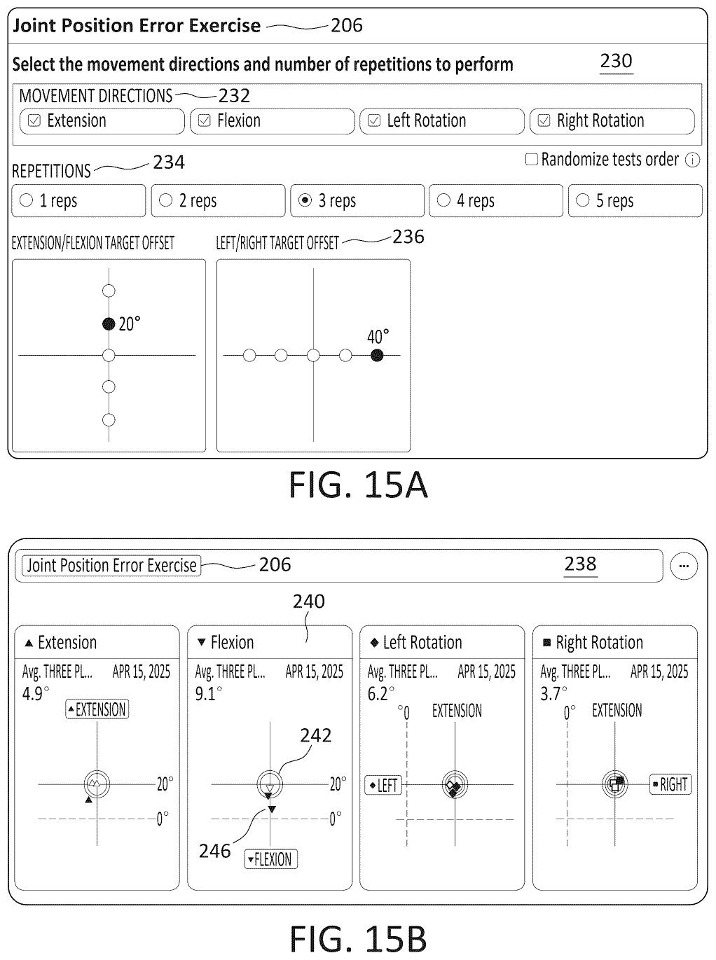

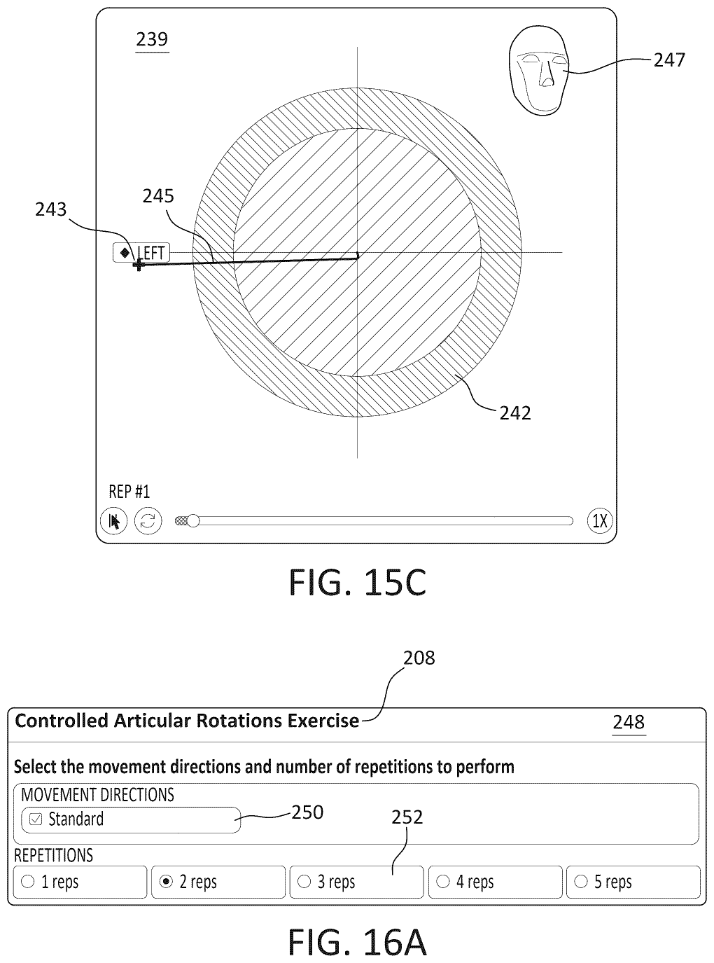

FIG. 15A illustrates an exemplary user interface 230 for the joint position error exercise 206. The joint position error exercise 206 is designed to improve proprioception (i.e., joint position sense) of the cervical spine. The user practices relocating to a starting, neutral head position after active movement with eyes closed. The joint position error exercise 206 can be performed from one or more movement directions 232, including flexion, extension, left-right rotation, and configured with one or more offset degree settings 236. The offset degree settings 236 are offset from a neutral position, i.e., instead of starting in and attempting to relocate to a neutral position after an active movement, the user first moves to an offset position and attempts to relocate to this position after an active movement. Like the user interface 212 for the Butterfly test 204, the user interface 230 is provided with a selectable repetition option 234. During the joint position error exercise 206, the user is prompted to slowly move (e.g., between extension and flexion) away from and return to a starting position (neutral or offset) a selected number of times. The user is instructed to keep their eyes closed, except when returning to the starting position, and the assessment application 112 will produce a sound (or vibration with the headset or tracking device 104) for when the user needs to open and close their eyes.

FIG. 15B illustrates an exemplary user interface 238 displaying results of the joint position error exercise 206. The user interface 238 can present one or more diagrams 240 that illustrates the performance of the user for the joint position error exercise 206. The number of diagrams 240 is dependent on the number of selected movement directions 232. Each diagram 240 can illustrate an exemplary icon 242 that depicts a target 242 for scoring recorded points 246 of a recorded joint position of the user. In an embodiment, when the exercise 206 is performed with an offset setting 236, the offset setting 236 could be indicated in the diagram 240. In an embodiment, a larger target 242 represents a greater distribution in recorded points 246. The target 242 and the points 246 can be color-coded to more easily indicate a favorable or less favorable score of the exercise 206 for the user's performance.

FIG. 15C illustrates a playback user interface 239 for graphically presenting the performance of the user for the joint position error exercise 206. Like the diagram 240 for displaying the results of the joint position error exercise 206, the playback user interface 239 shows the target 242 and can also show movement of a moveable cursor 243 controlled by the user during the joint position error exercise 206. The playback user interface 239 can also show a recorded path 245 of the user's performance. For traditional joint position error tests, which are typically performed with a laser and piece of paper on a wall, clinicians have not been able to review the path or movement of the user made during the exercise or assessment; instead, traditional joint position error tests only show a final position after the movement of the user. Advantageously, the playback user interface 239 can visualize the movement of the user for the joint position error exercise 206. Additionally, the playback user interface is provided with a 3D model 247 of the user's performance to represent a visualization of the 3D movement of the user's head (or another body part) during the joint position error exercise 206. One skilled in the art will recognize that the 3D model 247 can be applied to other exercises and assessments of the present disclosure. In an embodiment, the 3D model is synchronized with the recorded path 245 of the user's performance, wherein the 3D model depicts 3D movement and the recorded path 245 depicts 2D movement for the exercise and/or assessment.

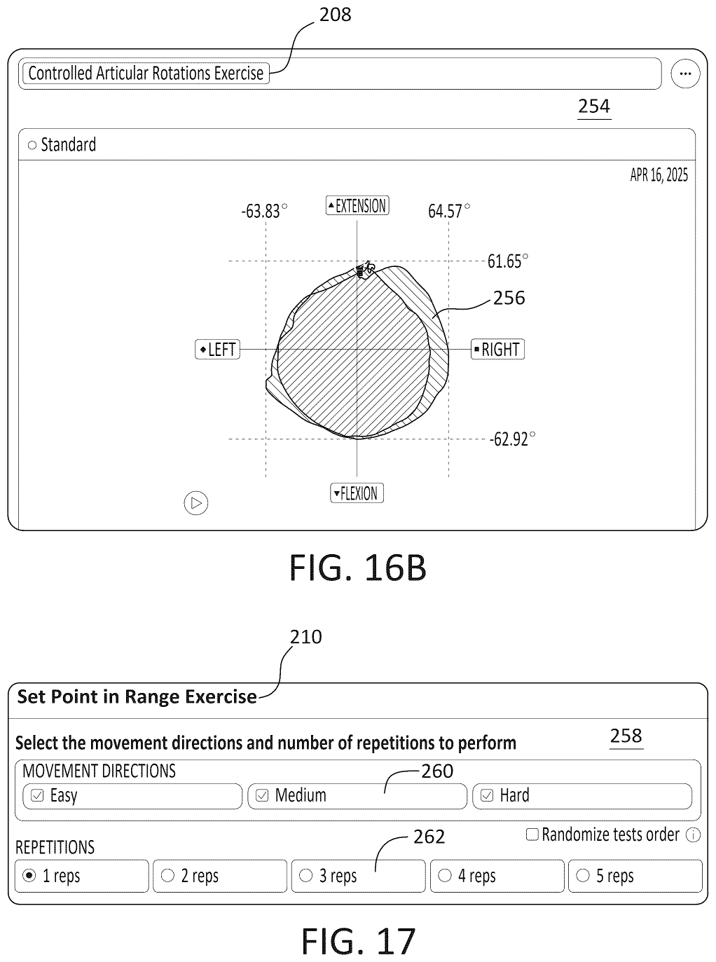

FIG. 16A illustrates an exemplary user interface 248 for the controlled articular rotations exercise 208. The exercise 208 includes slow and specific, controlled movements designed to improve the range of motion and joint health and adaptability. The exercise 208 is arranged for the user to actively move through a joint's full range of motion, specifically targeting the outer limits, to improve joint health. The user interface 248 includes a movement direction option 250, wherein the user can select a standard movement (e.g., circular rotation) for the user to perform during the exercise 208. Like the user interface 212 for the Butterfly test 204, the user interface 248 is provided with a selectable repetition option 252.

FIG. 16B illustrates an exemplary user interface 254 displaying results of the controlled articular rotations exercise 208. The user interface 254 can present a diagram 256 showing the recorded full range of motion in flexion/extension and left/right rotation performed by the user, including coupled motions, represented as a two-dimensional area. Multiple performances of the exercise 208 can be displayed simultaneously on the user interface 254 for comparison between exercise repetitions.

FIG. 17 illustrates an exemplary user interface 258 for the set point in range exercise 210. The set point in range exercise 210 is designed to relocate a natural position after an active movement or to actively relocate a position within a movement plane. For example, the set point in range exercise 210 can include one or more of the following tests: relocation to a natural head position, relocation to an offset (e.g., 30°) rotation position to the natural head position, hitting targets at a predetermined position, preset trunk rotation, figure-eight relocation, and figure-eight movement. The user interface 258 can include a movement direction option 260, which allows the user to select one or more directions (e.g., with different difficulty rankings). Like the user interface 212 for the Butterfly test 204, the user interface 258 is provided with a selectable repetition option 262.

The disclosed embodiments of the method and system 100 add versatility to rehabilitation for both impaired individuals (e.g., patients) and practitioners. The system 100 advantageously offers remote capability to accommodate telemedicine rehabilitation applications. Patients may carry out clinically verified assessment tests (or assessments) and exercises (or activities) in the comfort of their own homes. This, in turn, reduces the number of face-to-face clinic visits needed to ensure therapeutic effectiveness. The system 100 also adds versatility for the patient by reducing the cost associated with outpatient physical therapy. The cloud storage capability (e.g., network 124) allows clinicians to remotely access patient data, easily review sequential therapy sessions to assess patient progression or regression and modify the therapy regimens in response to patient performance. This versatility for patients to perform their therapy at home and the ability for clinicians to track these at-home therapy sessions may also improve patient compliance. The regular, consistent movement of an impaired joint in a controlled manner can lead to better patient outcomes. Thus, when patients increase their level of compliance to prescribed therapy and perform their therapy more frequently (i.e., as a consequence from implementing a simplified, accessible system 100 with enhanced patient education) there is an increased likelihood that their therapy will remediate their impairment and relieve their pain. Moreover, as the disclosed solution adds automation to the respective physical therapy session, the burden imposed on the clinician to iteratively and manually complete assessment steps is reduced. Thus, more time is provided to the clinician for data interpretation and rehabilitation outlining.

### Enumerated Embodiments and Examples of Systems and Methods for an Instructional Assessment Interface

Clause 1. A system for evaluating a condition of a user using an assessment application, the system comprising:
a first computing unit having the assessment application, a processor, one or more hardware storage devices, and a display for presenting the assessment application to the user, wherein the assessment application includes an instructional interface having at least one assessment with at least one activity for the user to perform;
a tracking device communicatively connected to the first computing unit and configured to obtain kinematic data corresponding to the user in a three-dimensional space during the at least one assessment;
wherein the one or more hardware storage devices store instructions that are executable by the system to:
   initiate the assessment application;
   determine a starting point for the at least one assessment based on the kinematic data obtained by the tracking device;
   detect enactment of an activity being performed by the user during the at least one assessment;
   determine an endpoint for the activity; and
   generate a report based on performance of the user for the at least one assessment;
wherein the instructional interface is configured to present at least one instructional image on the display, the at least one instruction image corresponding to at least one activity of the at least one assessment;
wherein the instructional interface is configured to depict an instructional image presented on the display emphasizing the activity being actively performed by the user.

Clause 2. The system according to clause 1, wherein, following automatic determination of the endpoint for the activity, the instructions are further executable to: detect a second activity being performed by the user;
foreground a second instructional image corresponding to the second activity; and
automatically determine a second endpoint for the second activity.

Clause 3. The system according to clause 1 or 2, wherein the at least one assessment is directed to evaluating at least one of range of motion, proprioception, balance, neuromuscular control, sensorimotor control, oculomotor control, pupillary response, sensorimotor integration, eye-head-neck coordination, vestibular function, cognition, and strength.

Clause 4. The system according to any one of clauses 1-3, wherein the at least one activity includes one or more of the following:
at least one of flexion, extension, rotation, abduction, adduction, circumduction, elevation, depression, pronation, supination, protraction, and retraction using a body portion of the user;
at least one of a static balance test and a dynamic balance test; and
a direct light reflex, a consensual light reflex, an accommodation reflex, and eye movement using one or more eyes of the user.

Clause 5. The system according to any one of clauses 2-4, wherein the instructional image is different from the second instructional image.

Clause 6. The system according to any one of clauses 2-5, wherein the instructional image and second instructional image are simultaneously displayed on the display.

Clause 7. The system according to any one of clauses 2-6, wherein the instructional image is muted when the second instructional image is foregrounded.

Clause 8. The system according to any one of clauses clause 1-7, wherein the at least one assessment includes a first assessment and a second assessment provided by the assessment application, the first and second assessments arranged to be concurrently performed by the user;

wherein the first assessment and second assessment each obtain one or more functional evaluations of range of motion, proprioception, balance, neuromuscular control, sensorimotor control, oculomotor control, pupillary response, sensorimotor integration, eye-head-neck coordination, vestibular function, cognition, and strength, the one or more functional evaluations of the first assessment being different from the one or more functional evaluations of the second assessment.

Clause 9. The system according to any one of clauses 2-8, wherein the instructional image is replaced by the second instructional image after the second activity is detected as being performed by the user.

Clause 10. The system according to any one of clauses clause 1-9, wherein the instructional interface is arranged to display or broadcast at least one performance indicator along with the at least one instructional image in real time during performance of the at least one activity.

Clause 11. A method for evaluating a condition of a user using a computing unit having an assessment application, a processor, one or more hardware storage devices, and a display for presenting the assessment application to the user, wherein the assessment application includes an instructional interface and is arranged to record physical assessment performance of the user during at least one assessment completed with the instructional interface, the method comprising:
initiating the assessment application;
using a tracking device connected to the assessment application, obtaining kinematic data corresponding to the user in a three-dimensional space;
automatically determining a starting point for the at least one assessment based on the kinematic data;
displaying at least one instructional image corresponding to at least one activity of the at least one assessment;
detecting a first activity being performed by the user;
foregrounding a first instructional image corresponding to the first activity being actively performed by the user;
automatically determining a first endpoint for the first activity; and
generating a report based on performance of the user for the at least one assessment.

Clause 12. The method according to clause 11, comprising the following steps following automatically determining the first endpoint for the first activity: detecting a second activity being performed by the user;
foregrounding a second instructional image corresponding to the second activity; and
automatically determining a second endpoint for the second activity.

Clause 13. The method according to clause 11 or 12, wherein the at least one assessment is directed to evaluating at least one of range of motion, proprioception, balance, neuromuscular control, sensorimotor control, oculomotor control, pupillary response, sensorimotor integration, eye-head-neck coordination, vestibular function, cognition, and strength.

Clause 14. The method according to clause 12 or 13, wherein the first instructional image is different from the second instructional image.

Clause 15. The method according to any one of clauses 12-14, wherein the first instructional image and second instructional image are simultaneously presented on a display.

Clause 16. The method according to any one of clauses 12-15 further comprising simultaneously muting the first instructional image while foregrounding the second instructional image.

Clause 17. The method according to any one of clauses 11-16 further comprising simultaneously executing a first assessment and a second assessment;
wherein the first assessment is directed to evaluating the condition and obtaining one or more first evaluations of range of motion, proprioception, balance, neuromuscular control, sensorimotor control, oculomotor control, pupillary response, sensorimotor integration, eye-head-neck coordination, vestibular function, cognition, and strength;
wherein the second assessment is directed to evaluating the condition and obtaining one or more second evaluations of range of motion, proprioception, balance, neuromuscular control, sensorimotor control, oculomotor control, pupillary response, sensorimotor integration, eye-head-neck coordination, vestibular function, cognition, and strength;
wherein the one or more first evaluations are different from the one or more second evaluations.

Clause 18. The method according to clause 17, wherein the first assessment and the second assessment include at least one concurrent activity to obtain the one or more first evaluations and the one or more second evaluations.

Clause 19. The method according to any one of clauses 12-18 further comprising replacing the first instructional image with the second instructional image after the second activity is detected as being performed by the user.

Clause 20. A system for evaluating a condition of a user using an assessment application, the system comprising:
a first computing unit having the assessment application, a processor, one or more hardware storage devices, and a display for presenting the assessment application to the user, wherein the assessment application includes an instructional interface having at least one assessment with at least one activity for the user to perform;
a tracking device communicatively connected to the first computing unit and configured to observe physical assessment performance of the user and obtain kinematic data corresponding to the user in a three-dimensional space during the at least one assessment;
wherein the one or more hardware storage devices store instructions that are executable by the system to:
   initiate the assessment application;
   automatically determine a starting point for a movement control assessment based on the kinematic data;
   display at least one instructional image or text corresponding to at least one activity of the movement control assessment on the display;
   detect a first activity being performed by the user, the first activity including a moving indicator that follows a computer-generated path and a cursor controllable by the user for performing the first activity;
   visually indicate movement accuracy performance of the user during execution of the first activity by a change in color of the moving indicator and based on deviation of the cursor from the moving indicator; and automatically determine an endpoint corresponding to the first activity; and generate a report based on performance of the user for the at least one assessment.

It is to be understood that even though numerous characteristics and advantages of various embodiments of the present disclosure have been outlined in the foregoing description, together with details of the structure and function of various embodiments thereof, this detailed description is illustrative only, and changes may be made in detail, especially in matters of structure and arrangements of parts within the principles of the present disclosure to the full extent indicated by the broad general meaning of the terms in which the appended claims are expressed.

Various disclosed features of the present application are interchangeable. Besides the variations described herein, other known equivalents for each feature can be mixed and matched to develop systems and methods for instructional interfaces assessment applications and exercise modules under principles of the present disclosure. The features described herein may be adapted to other methods and types of devices applications for evaluating conditions of a user.

It is intended that the present disclosure should not be limited by the disclosed embodiments described above and may be extended to other applications that may employ the features described herein.

1. A system for evaluating a condition of a user using an assessment application, the system comprising:
a first computing unit having the assessment application, a processor, one or more hardware storage devices, and a display for presenting the assessment application to the user, wherein the assessment application includes an instructional interface having at least one assessment with at least one activity for the user to perform;
a tracking device communicatively connected to the first computing unit and configured to obtain kinematic data corresponding to the user in a three-dimensional space during the at least one assessment;
wherein the one or more hardware storage devices store instructions that are executable by the system to:
initiate the assessment application;
determine a starting point for the at least one assessment based on the kinematic data obtained by the tracking device;
detect enactment of an activity being performed by the user during the at least one assessment;
determine an endpoint for the activity; and
generate a report based on performance of the user for the at least one assessment;
wherein the instructional interface is configured to present at least one instructional image on the display, the at least one instructional image corresponding to at least one activity of the at least one assessment;
wherein the instructional interface is configured to depict an instructional image presented on the display emphasizing the activity being actively performed by the user.

2. The system according to claim 1, wherein, following automatic determination of the endpoint for the activity, the instructions are further executable to:
detect a second activity being performed by the user;
foreground a second instructional image corresponding to the second activity; and
automatically determine a second endpoint for the second activity.

3. The system according to claim 1, wherein the at least one assessment is directed to evaluating at least one of range of motion, proprioception, balance, neuromuscular control, sensorimotor control, oculomotor control, pupillary response, sensorimotor integration, eye-head-neck coordination, vestibular function, cognition, and strength.

4. The system according to claim 1, wherein the at least one activity includes one or more of the following:
at least one of flexion, extension, rotation, abduction, adduction, circumduction, elevation, depression, pronation, supination, protraction, and retraction using a body portion of the user;
at least one of a static balance test and a dynamic balance test; and
a direct light reflex, a consensual light reflex, an accommodation reflex, and eye movement using one or more eyes of the user.

5. The system according to claim 2, wherein the instructional image is different from the second instructional image.

6. The system according to claim 2, wherein the instructional image and second instructional image are simultaneously displayed on the display.

7. The system according to claim 2, wherein the instructional image is muted when the second instructional image is foregrounded.

8. The system according to claim 1, wherein the at least one assessment includes a first assessment and a second assessment provided by the assessment application, the first and second assessments arranged to be concurrently performed by the user;
wherein the first assessment and second assessment each obtain one or more functional evaluations of range of motion, proprioception, balance, neuromuscular control, sensorimotor control, oculomotor control, pupillary response, sensorimotor integration, eye-head-neck coordination, vestibular function, cognition, and strength, the one or more functional evaluations of the first assessment being different from the one or more functional evaluations of the second assessment.

9. The system according to claim 2, wherein the instructional image is replaced by the second instructional image after the second activity is detected as being performed by the user.

10. The system according to claim 1, wherein the instructional interface is arranged to display or broadcast at least one performance indicator along with the at least one instructional image in real time during performance of the at least one activity.

11. A method for evaluating a condition of a user using a computing unit having an assessment application, a processor, one or more hardware storage devices, and a display for presenting the assessment application to the user, wherein the assessment application includes an instructional interface and is arranged to record physical assessment performance of the user during at least one assessment completed with the instructional interface, the method comprising:
initiating the assessment application;
using a tracking device connected to the assessment application, obtaining kinematic data corresponding to the user in a three-dimensional space;
automatically determining a starting point for the at least one assessment based on the kinematic data;
displaying at least one instructional image corresponding to at least one activity of the at least one assessment;
detecting a first activity being performed by the user;
foregrounding a first instructional image corresponding to the first activity being actively performed by the user;
automatically determining a first endpoint for the first activity; and
generating a report based on performance of the user for the at least one assessment.

12. The method according to claim 11, comprising the following steps following automatically determining the first endpoint for the first activity:
detecting a second activity being performed by the user;
foregrounding a second instructional image corresponding to the second activity; and
automatically determining a second endpoint for the second activity.

13. The method according to claim 11, wherein the at least one assessment is directed to evaluating at least one of range of motion, proprioception, balance, neuromuscular control, sensorimotor control, oculomotor control, pupillary response, sensorimotor integration, eye-head-neck coordination, vestibular function, cognition, and strength.

14. The method according to claim 12, wherein the first instructional image is different from the second instructional image.

15. The method according to claim 12, wherein the first instructional image and second instructional image are simultaneously presented on the display.

16. The method according to claim 12 further comprising simultaneously muting the first instructional image while foregrounding the second instructional image.

17. The method according to claim 11 further comprising simultaneously executing a first assessment and a second assessment;
wherein the first assessment is directed to evaluating the condition and obtaining one or more first evaluations of range of motion, proprioception, balance, neuromuscular control, sensorimotor control, oculomotor control, pupillary response, sensorimotor integration, eye-head-neck coordination, vestibular function, cognition, and strength;
wherein the second assessment is directed to evaluating the condition and obtaining one or more second evaluations of range of motion, proprioception, balance, neuromuscular control, sensorimotor control, oculomotor control, pupillary response, sensorimotor integration, eye-head-neck coordination, vestibular function, cognition, and strength;
wherein the one or more first evaluations are different from the one or more second evaluations.

18. The method according to claim 17, wherein the first assessment and the second assessment include at least one concurrent activity to obtain the one or more first evaluations and the one or more second evaluations.

19. The method according to claim 12 further comprising replacing the first instructional image with the second instructional image after the second activity is detected as being performed by the user.

20. A system for evaluating a condition of a user using an assessment application, the system comprising:
a first computing unit having the assessment application, a processor, one or more hardware storage devices, and a display for presenting the assessment application to the user, wherein the assessment application includes an instructional interface having at least one assessment with at least one activity for the user to perform;
a tracking device communicatively connected to the first computing unit and configured to observe physical assessment performance of the user and obtain kinematic data corresponding to the user in a three-dimensional space during the at least one assessment;
wherein the one or more hardware storage devices store instructions that are executable by the system to:
initiate the assessment application;
automatically determine a starting point for a movement control assessment based on the kinematic data;
display at least one instructional image or text corresponding to at least one activity of the movement control assessment on the display;
detect a first activity being performed by the user, the first activity including a moving indicator that follows a computer-generated path and a cursor controllable by the user for performing the first activity;
visually indicate movement accuracy performance of the user during execution of the first activity by a change in color of the moving indicator and based on deviation of the cursor from the moving indicator; and
automatically determine an endpoint corresponding to the first activity; and
generate a report based on performance of the user for the at least one assessment.
